(19) United States
(12) Patent Application Publication
(10) Pub. No.: US 2023/0203339 A1
(43) Pub. Date: Jun. 29, 2023

(54) Amino Acid Helical Array Film and Preparation Method Thereof

(71) Applicant: ZJU-Hangzhou Global Scientific and Technological Innovation Center, Hangzhou (CN)

(72) Inventors: Kai Tao, Hangzhou (CN); Haoran Wu, Hangzhou (CN); Jiahao Zhang, Hangzhou (CN); Bin Yu, Hangzhou (CN)

(73) Assignee: ZJU-Hangzhou Global Scientific and Technological Innovation Center, Hangzhou (CN)

(21) Appl. No.: 18/117,016

(22) Filed: Mar. 3, 2023

(30) Foreign Application Priority Data
Mar. 3, 2022 (CN) 202210201463.9

(51) Int. Cl. C09D 177/04; C23C 14/24; C23C 14/12

(52) U.S. Cl. CPC C09D 177/04; C23C 14/24; C23C 14/12

(57) ABSTRACT

The present disclosure discloses an amino acid helical array film and a preparation method thereof. The amino acid helical array film comprises a substrate and an amino acid helical array uniformly deposited on the substrate. Each amino acid helix is obtained by self-assembling an amino acid with a modifying group. The amino acid is selected from one or more of twenty common natural amino acids or adjacent isomers thereof. The modifying group comprises an N-terminal protecting group and a C-terminal protecting group, wherein the N-terminal protecting group is selected from one or more of carbobenzoxy, a lipid group, t-butoxycarbonyl, and 9-fluorenylmethoxycarbonyl, and the C-terminal protecting group is selected from one or more of nitrophenyl ester, a lipoxy group, and an acylamino group.

### TECHNICAL FIELD

The present disclosure relates to the technical field of bio-organic self-assembled superstructural materials and surface functional structures, and particularly relates to an amino acid helical array film and a preparation method thereof.

### BACKGROUND ART

An environmentally friendly and biocompatible self-assembled superstructure and a functional device manufactured based on a design of the superstructure would revolutionize lifestyles of human However, the traditional inorganic or organic self-assembled superstructure manufacturing technology cannot provide a perfect biocompatibility, has a complex preparation condition or performance regulation process (requiring a harsh temperature and pressure or other extreme environmental conditions), such that a requirement of a fusion interaction of a bio-device interface is difficult to meet. Bio-organic micromolecules represented by amino acids have excellent material characteristics such as wide raw material source, high design flexibility, simple preparation, and have inherent biocompatibility. Therefore, by utilizing intelligent assembly, interface modification, and array integration of amino acids and other bio-organic micromolecules, a superstructure system with various morphological sizes and adjustable performances may be prepared, and various bio-organic functional devices can be designed and fabricated, which has a wide application prospect in a bio-device interface interaction. The array integration may also greatly expand the morphologies and performances of a single bio-organic self-assembly, can be prepared in a large scale, is convenient for design and manufacture of subsequent devices, and becomes a current frontier of science and technology. Therefore, the bio-organic self-assembly system is further assembled in an array, such that physicochemical performances of a single assembly can be integrated and commercialization is expected to be realized. Therefore, more and more researches are focused on the array arrangement of the bio-organic micro-molecule self-assembly system.

Currently, an array integration process of the bio-organic micromolecule self-assembled superstructure mainly comprises a horizontal deposition by a drop-coating method, an electric/magnetic field auxiliary arrangement, an external force traction auxiliary arrangement or a template auxiliary arrangement and the like. The horizontal deposition by a drop-coating method is only suitable for a spherical self-assembly. The electric/magnetic field auxiliary arrangement requires that the self-assembly has a polarity or magnetic performance, the external force traction auxiliary arrangement and the template auxiliary arrangement have a relatively low precision. An array morphology is limited by a processing technology. In comparison, a physical vapor deposition can directly sublimate and deposit a solid-state bio-organic material on a surface of a substrate to obtain a self-assembled array structure with a regular morphology, use of a solvent is avoided, and the method has a unique advantage of forming the large-scale array integration from the molecular self-assembly in one step. However, the technology of preparing the bio-organic micromolecule self-assembled array by the physical vapor deposition has rarely been reported so far. Currently, it is only known that an array film structure may be prepared by an individual short peptide (such as diphenylalanine) in the physical vapor deposition (Nat. Nanotechnol. 4, 849-854 (2009); Nano Lett. 9, 3111-3115(2009)). However, the single self-assembly in the array film prepared by the technical solution only stays in a longitudinal vertical direction, and has a single structure, and a poor shape and property regulation and control performance. Besides, a synthesis cost of a peptide is relatively high and large-scale popularization and use of the self-assembled array film are limited. Therefore, related preparation process method, array morphology regulation and control, physicochemical performance characterization, use and the like for preparing a complex topological array film structure by using bio-organic micromolecules with simple structures, especially a helical array structure with a chiral characteristic, are not reported yet.

### SUMMARY OF THE INVENTION

Aiming at the problems in the prior art, the present disclosure discloses an amino acid helical array film and a preparation method thereof. The preparation process is simple and adjustable, and has high repeatability. An amino acid self-assembly is uniformly arranged in a helical array on the prepared amino acid film. Besides, all the helixes have a uniform rotation direction and the rotation direction can be adjusted. A morphological characteristic of the amino acid film is very important to physicochemical properties (such as optical, electrical, and mechanical performances) of the film, and design, development, and application of functional devices in related fields.

Specific technical solutions are as follows:

An amino acid helical array film comprises a substrate and an amino acid helical array uniformly deposited on the substrate;
each amino acid helix is obtained by self-assembling an amino acid with a modifying group;
the amino acid is selected from glycine, alanine, valine, leucine, isoleucine, methionine, proline, tryptophan, serine, tyrosine, cysteine, phenylalanine, asparagine, glutamine, threonine, aspartic acid, glutamic acid, lysine, arginine, and histidine in twenty common natural amino acids (α-amino acids), or one or more of adjacent isomers of the above natural amino acids; and
the modifying group comprises an N-terminal protecting group and a C-terminal protecting group, wherein the N-terminal protecting group is selected from one or more of carbobenzoxy, a lipid group, t-butoxycarbonyl, and 9-fluorenylmethoxycarbonyl, and the C-terminal protecting group is selected from one or more of nitrophenyl ester, a lipoxy group, and an acylamino group.

The adjacent isomers refer to respective β-amino acids corresponding to the twenty natural amino acids, which closely resemble properties of the respective corresponding natural amino acids (α-amino acids).

The present disclosure discloses an amino acid self-assembly film for the first time, wherein the film is uniformly deposited on a substrate and an amino acid self-assembly in the film is uniformly distributed in a helical array. Besides, the amino acid helical array has a uniform rotation direction in a clockwise or an anticlockwise manner. The rotation direction of the obtained amino acid helical array may be regulated and controlled by adjusting chirality of an amino acid as a raw material.

Specifically, when the amino acid in the raw material is L-type, the prepared amino acid helical array rotates clockwise; and when the amino acid used is D-type, the prepared amino acid helical array rotates anticlockwise.

The amino acid helix has a diameter range of 300-650 μm. Besides, the diameter distribution is narrow and the size is uniform.

The amino acid helical array film prepared by the present disclosure has no special requirements on a deposited substrate. The substrate may be made of metal, glass or polymer, and hydrophilic, hydrophobic, electrically conductive, thermally conductive, insulating, flexible, rigid, transparent, semitransparent, and opaque, and made of an inorganic material or an organic material. It can be seen that the amino acid helical array film has an excellent universality for the substrate.

The size of the substrate may be adjusted arbitrarily according to a size of a desired array.

Preferably, the amino acid is selected from L-phenylalanine or D-phenylalanine, the N-terminal protecting group is selected from t-butoxycarbonyl, and the C-terminal protecting group is selected from nitrophenyl ester.

The present disclosure further discloses a preparation method of the amino acid helical array film, wherein a physical vapor deposition is used and specifically comprises:
placing an amino acid raw material with a modifying group in an evaporation boat of a reaction chamber and obtaining a self-assembled amino acid helical array film by deposition on a surface of the substrate through a vacuum evaporation coating method.

The amino acid is selected from glycine, alanine, valine, leucine, isoleucine, methionine, proline, tryptophan, serine, tyrosine, cysteine, phenylalanine, asparagine, glutamine, threonine, aspartic acid, glutamic acid, lysine, arginine, histidine, or one or more of adjacent isomers of the above amino acids; and
the modifying group comprises an N-terminal protecting group and a C-terminal protecting group, wherein the N-terminal protecting group is selected from carbobenzoxy, a lipid group, t-butoxycarbonyl, and 9-fluorenylmethoxycarbonyl, and the C-terminal protecting group is selected from nitrophenyl ester, a lipoxy group, and an acylamino group.

Preferably, the amino acid raw material with a modifying group is selected from t-butyloxy carboryl-L-phenylalanine-nitrophenyl ester or t-butyloxy carboryl-D-pheny lalanine-nitrophenyl ester.

The present disclosure uses a physical vapor deposition process, specifically a vacuum evaporation coating method. A specific amino acid with a modifying group is used as a raw material and an amino acid helical array film structure is prepared for the first time. Besides, a rotation direction of the prepared amino acid helical array may be regulated and controlled according to chirality of the selected raw material amino acid. The prepared amino acid helical array has different characteristics according to different modifying groups. A diameter of the prepared amino acid helical array is adjusted by regulating and controlling deposition process parameters. In the preparation process, selection of raw materials and a distance between an evaporation boat and a substrate are particularly critical. Tests show that if the amino acid is not modified, taking phenylalanine as an example, and if only L-phenylalanine or D-phenylalanine is used as a raw material, the prepared amino acid self-assembly film does not have a special morphology of a helical array. If the modified L-phenylalanine and the modified D-phenylalanine are blended to be used as a raw material, a regular and uniform amino acid helical array film cannot be obtained as well. In addition, the distance between the evaporation boat and the substrate also determines whether a target product may be obtained through deposition. The distance is controlled to be 1-5 cm in the present disclosure (a vapor deposition device needs to be customized, but except that the distance between the evaporation boat and the substrate is a special size, other parts of the device and sizes thereof are not different from those of a common vapor deposition device ZFS-500 in the prior art), such that the amino acid helical array film can be obtained through a high-efficiency deposition. However, if a conventional vapor deposition device is used (the distance between the evaporation boat and the substrate is relatively large, generally 10-40 cm), no obvious self-assembled film structure can be observed on the substrate within 48 h.

The inventor further conducts comparative experiments. The same raw materials as in the present disclosure are used, but a solvent evaporation method is used to self-assemble amino acids. As a result, the prepared film structure does not have helicity. The results indicate that both the special raw material selection and the specific physical vapor deposition process in the present disclosure are essential.

The vacuum evaporation coating method is as follows:
vacuumizing a reaction chamber until a vacuum degree is less than or equal to 5×10⁻⁶ mbar, firstly performing heating to a sublimation temperature of the amino acid raw material with a modifying group, then performing heating to a highest temperature, and preserving the temperature for a period of time;
the highest temperature is 200-220° C.; and
total time from the heating to the sublimation temperature to the end of the highest temperature preserving is recorded as deposition time, selected from 15-60 min.

Tests show that a morphology of the prepared amino acid helical array may be further regulated and controlled by regulating and controlling deposition time and the amount of raw materials in the vacuum evaporation coating process.

When the deposition time is too short, the prepared amino acid helical array has a relatively low assembled density. But when the deposition time is too long, a helix structure of the prepared amino acid helical array cannot be observed significantly due to too high assembly density.

Preferably, the deposition time is 30-60 min. The amino acid helical array prepared under the preferable condition has a moderate density and an obvious helical structure. The helical array has a narrow diameter distribution and a more uniform size.

When the amount of the raw materials is too small or too large, the assembled density of the prepared amino acid helical array is changed, preferably, in the device used in the present disclosure, the mass of the added raw materials is 3-20 mg, more preferably 5-10 mg. The amino acid helical array prepared under the amount of the raw materials has a moderate density, an obvious helical structure, a narrow diameter distribution, and a more uniform size.

However, it should be noted that if the size of the device is changed, the amount of the raw materials can be adjusted adaptively according to a principle of the present disclosure. Besides, an adjustment of the quality of the raw materials still falls within the protection scope of the present disclosure.

To ensure that the deposited amino acid helical array is more uniform and adjustable, it is preferred that the deposition is performed by heating in stages, for example, taking a modified phenylalanine as a raw material:
a first-stage heating: setting 10 min for a room temperature rising to 60° C.; a second-stage heating: setting 10 min for a temperature of 60° C. rising to 160° C.; a third-stage heating: setting a period of time for a temperature of 160° C. rising to 220° C.; and a fourth-stage heating: maintaining a temperature at 220° C. for a period of time.

In the heating process, the deposition time starts from the third-stage heating to the end of the fourth-stage heating.

Compared with the prior art, the present disclosure has the following beneficial effects:

(1) The present disclosure prepares the helix-shaped bio-organic self-assembled array for the first time using a physical vapor deposition method, which is important for researching physical and chemical performances of the helix-shaped micro-nano array. The preparation process has a simple flow and a high system automation integration degree, is capable of realizing batch and large-scale preparation, has a good result repeatability, avoids use of a solvent, and has a controllable cost and less pollution.

(2) The prepared bio-organic helical array film prepared in the present disclosure is formed by self-assembling amino acids and has a special helical array morphology. The rotation direction of the bio-organic self-assembled helical array may be regulated and controlled through the chirality of the raw material amino acid. The morphology of the helical array may further be regulated and controlled through regulating a deposition process. The present disclosure is important for researching the physical and chemical performances of the helical arrays with different rotation directions, particularly optical, electrical, magnetic, and mechanical performances and the like.

### BRIEF DESCRIPTION OF THE DRAWINGS

FIG. 1 shows SEM images of the amino acid helical array prepared in example 1 at different magnification times;

FIG. 2 is a statistical distribution diagram of diameters of the amino acid helical array prepared in example 1;

FIG. 3 is a confocal microscopy morphological image of the amino acid helical array prepared in example 1;

FIG. 4 is a fluorescent microscopic image of the amino acid helical array prepared in example 1;

FIG. 5 is an SEM image of the amino acid helical array prepared in example 15;

FIG. 6 is an SEM image of the amino acid helical array prepared in example 17;

FIG. 7 is an SEM image of a film of L-phenylalanine prepared in comparative example 1 deposited on a glass substrate;

FIG. 8 is an SEM image of an amino acid film deposited on a glass substrate in comparative example 3 by a solvent evaporation method;

FIG. 9 shows SEM images of the amino acid helical array prepared in example 21 at different magnification times;

FIG. 10 is a statistical distribution diagram of diameters of the amino acid helical array prepared in example 21;

FIG. 11 is a confocal microscopy morphological image of the amino acid helical array prepared in example 21; and

FIG. 12 is an SEM image of the amino acid self-assembled array film prepared in comparative example 4.

### DETAILED DESCRIPTION OF THE INVENTION

The present disclosure is described in further detail below with reference to examples and comparative examples, but embodiments of the present disclosure are not limited thereto.

### Example 1

Firstly 5 mg of t-butoxycarbonyl-(L-)phenylalanine-nitrophenyl ester powder (Boc-(L)F-ONp) (Chem-imprex Int'l. Inc.) was weighed; the weighed t-butoxycarbonyl-(L-)phenylalanine-nitrophenyl ester powder was placed into an evaporation boat in a main generation chamber; a distance between a glass substrate (2 cm×2 cm) and an evaporation boat was adjusted and set to be 1.5 cm; a door of the main generation chamber was closed to form a closed environment after the completion; an air extracting pump was turned on to pre-evacuate the main generation chamber to 0.1 mbar, after an air tightness of the main generation chamber was checked, the main generation chamber was vacuumized to about 1×10⁻⁵ mbar using the air extracting pump, and then a molecular pump was turned on to further vacuumize the main generation chamber until a vacuum degree is less than or equal to 5×10⁻⁶ mbar; and after the vacuum degree reaches the requirement, a temperature control procedure and a deposition time control procedure were set on a temperature control panel of a main console. In the present example, four stages of heating were performed: a first-stage heating: 10 min was set for a room temperature rising to 60° C.; a second-stage heating: 10 min was set for a temperature of 60° C. rising to 160° C.; a third-stage heating: 20 min was set for a temperature of 160° C. rising to 220° C.; and a fourth-stage heating: a temperature was maintained at 220° C.°C. for 15 min; after the deposition is completed, a baffle plate was driven to stop the deposition; a circulating water cooling device was turned on to cool the evaporation boat and the main generation chamber; and after cooling to a room temperature, an amino acid self-assembled helical array film was obtained on a glass substrate.

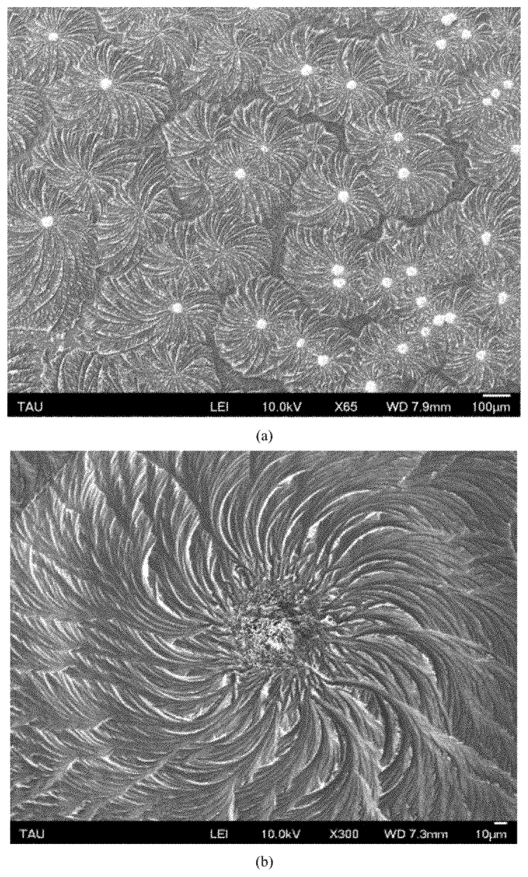

FIG. 1 showed SEM images of the amino acid self-assembled helical array prepared in the example at different magnification times. It can be seen by observing (a) that the prepared amino acid helical array was uniformly distributed on the substrate and had a consistent rotation direction in a clockwise manner. It can be seen by observing (b, c) that the helical array was arranged and assembled by a plurality of amino acid needle-shaped crystal fibers.

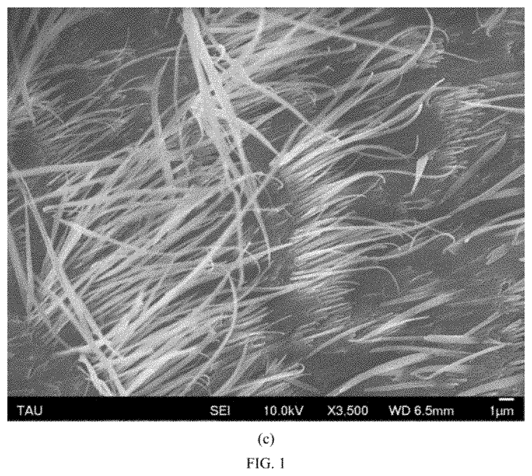

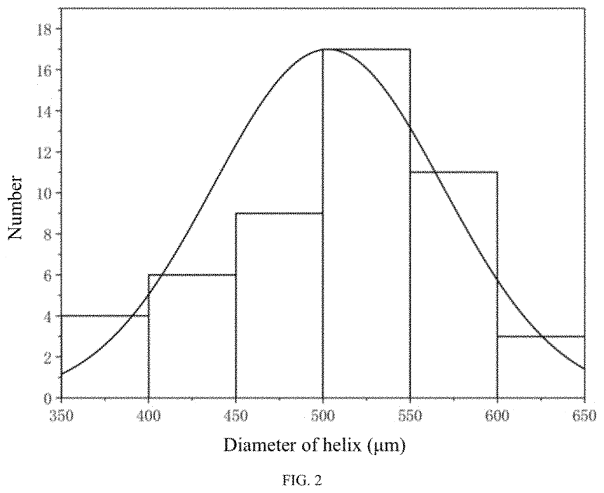

FIG. 2 was a statistical distribution diagram of diameters of the amino acid self-assembled helical array prepared in the example. The statistical result showed that the amino acid helical array had a statistical average diameter of 550+/−36 μm, a narrow diameter distribution, and a relatively uniform diameter.

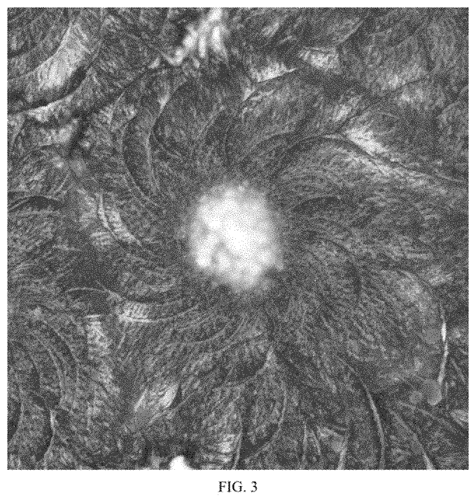

FIG. 3 was a confocal microscopy image of the amino acid helical array prepared in the example. It can be seen by combining FIG. 1 and the figure that self-assembled crystal fibers at a center of the helical array were tightly clustered and then spread out in an arrangement along a clockwise helix rotation direction.

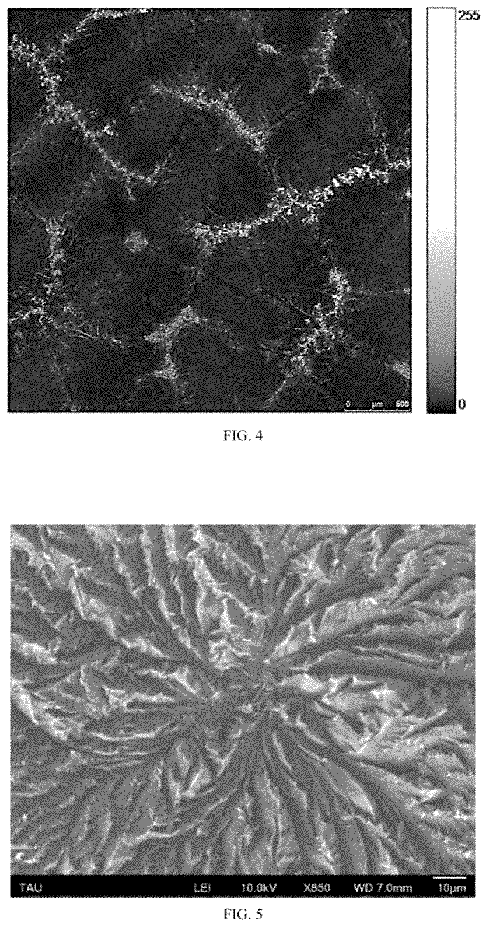

FIG. 4 was a fluorescent microscopic image of the amino acid helical array prepared in the example. Under a fluorescence microscope, the amino acid helical array may be observed to have an optical waveguide effect, and fluorescence may be transmitted to an outer layer through crystal fibers in the array, such that the helixes had higher brightness at edges and were relatively dark in the middle.

### Examples 2-14

The preparation process was basically the same as that in example 1. A difference was only that the substrate was replaced by a silicon wafer, a silicon dioxide sheet, a mica sheet (inorganic insulating substrate), a copper sheet, an aluminum sheet, a gold film (electrically conductive and thermally conductive metal substrate), an ITO thermally conductive glass sheet (inorganic electrically conductive substrate), a graphite sheet (hydrophobic electrically conductive substrate), an aluminum foil, a gold foil, silver-plated Polyvinylidene fluoride (flexible electrically conductive substrate) and PlantDesignManagementsystem, and Polyvinyl alcohol (flexible insulating substrate).

The morphology of the amino acid helical array prepared in each of the above examples was substantially similar to that in example 1, which indicated that the preparation process of the amino acid helical array disclosed in the present disclosure had universality for the substrates of various materials and properties.

### Example 15

The preparation process was basically the same as that in example 1. A difference only lied in that deposition time was different and shortened to 15 min. Specifically, a first-stage heating: 10 min was set for a room temperature rising to 60° C.; a second-stage heating: 10 min was set for a temperature of 60° C. rising to 160° C.; a third-stage heating: 10 min was set for a temperature of 160° C. rising to 220° C.; and a fourth-stage heating: a temperature was maintained at 220° C. for 5 min.

FIG. 5 was an SEM image of the amino acid helical array prepared in the example. It can be seen from the figure that a rotation direction of the amino acid helical array prepared by the example was consistent with that in example 1 in a clockwise manner. But the helical array had a relatively low assembling density.

### Example 16

The preparation process was basically the same as that in example 1. A difference only lied in that deposition time was different and adjusted to 60 min. Specifically, a first-stage heating: 10 min was set for a room temperature rising to 60° C.; a second-stage heating: 10 min was set for a temperature of 60° C. rising to 160° C.; a third-stage heating: 30 min was set for a temperature of 160° C. rising to 220° C.; and a fourth-stage heating: a temperature was maintained at 220° C. for 30 min.

Through SEM characterization, a morphology of the amino acid helical array prepared in the example was basically similar to that in example 1. The rotation direction was also consistent in a clockwise manner.

### Example 17

The preparation process was basically the same as that in example 1. A difference only lied in that deposition time was different and extended to 120 min. Specifically, a first-stage heating: 10 min was set for a room temperature rising to 60° C.; a second-stage heating: 10 min was set for a temperature of 60° C. rising to 160° C.; a third-stage heating: 60 min was set for a temperature of 160° C. rising to 220° C.; and a fourth-stage heating: a temperature was maintained at 220° C. for 60 min.

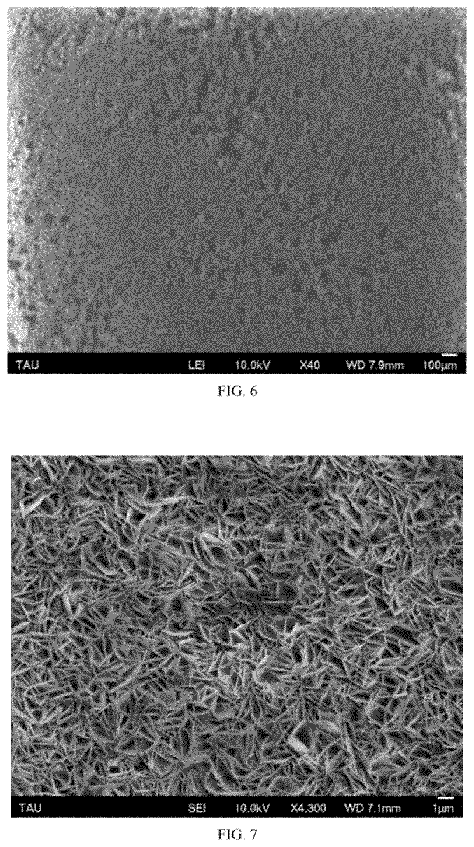

FIG. 6 was an SEM image of the amino acid helical array prepared in the example. It can be seen from the figure, the amino acid helical array prepared by the example had a relatively high density and no obvious helical structure.

It can be seen from the SEM images of the amino acid helical arrays prepared in comparative example 1 and examples 15-17, morphologies of the amino acid helical arrays may be adjusted by regulating and controlling the deposition time.

### Example 18

The preparation process was substantially the same as that in example 1. A difference only lied in that mass of the added t-butoxycarbonyl-(L-)phenylalanine-nitrophenyl ester powder varied. Specifically, the mass of the raw material was reduced to 0.5 mg.

Through SEM characterization, the amino acid helical array prepared in the example had a morphology substantially similar to that prepared in example 15.

### Example 19

The preparation process was substantially the same as that in example 1. A difference only lied in that mass of the added t-butoxycarbonyl-(L-)phenylalanine-nitrophenyl ester powder was adjusted to 10 mg.

Through SEM characterization, the amino acid helical array prepared in the example had a morphology substantially similar to that prepared in example 1.

### Example 20

The preparation process was substantially the same as that in example 1. A difference only lied in that mass of the added t-butoxycarbonyl-(L-)phenylalanine-nitrophenyl ester powder was increased to 50 mg.

Through SEM characterization, the amino acid helical array prepared in the example had a morphology substantially similar to that prepared in example 17.

It can be seen from the SEM images of the amino acid helical arrays prepared in comparative example 1 and examples 18-20, a morphology of the amino acid helical array may also be adjusted by regulating and controlling the mass of the raw material.

### Comparative Example 1

The preparation process was basically the same as that in example 1. A difference only lied in that L-phenylalanine of the same mass was used as a raw material.

FIG. 7 was an SEM image of L-phenylalanine deposited on a glass substrate. It can be seen from the SEM image, a film of a closely arranged plate-like crystal array prepared in the comparative example did not have a helical structure, which indicated that the amino acid helical array disclosed in the present disclosure must be prepared from terminal group-protected amino acids.

### Comparative Example 2

The preparation process was substantially the same as that in example 1. A difference only lied in that a device used in the comparative example was a conventional physical vapor deposition device with a model of ZFS-500. In the device, a distance between a substrate and an evaporation boat was 40 cm.

Tests showed that an amino acid helical array film cannot be successfully deposited on a substrate by a same deposition process using the conventional device, indicating that the amino acid helical array disclosed by the present disclosure must be prepared by a relatively small distance between the substrate and the evaporation boat.

### Comparative Example 3

The preparation process was basically the same as that in example 1. A difference only lied in that in the comparative example, an amino acid film was prepared by a solvent evaporation method, specifically: a hexafluoroisopropanol (HFIP) solution of Boc-(L)F-ONp was directly dripped on a glass substrate and an amino acid film was formed after the HFIP was evaporated.

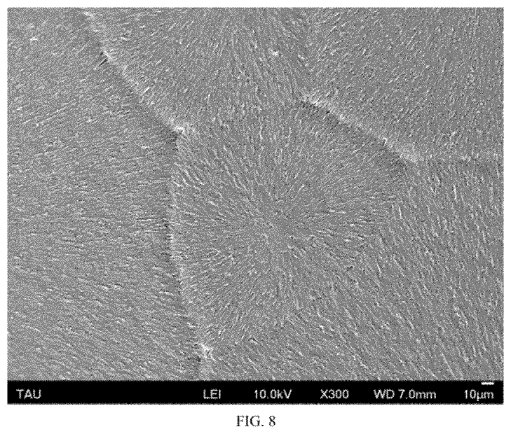

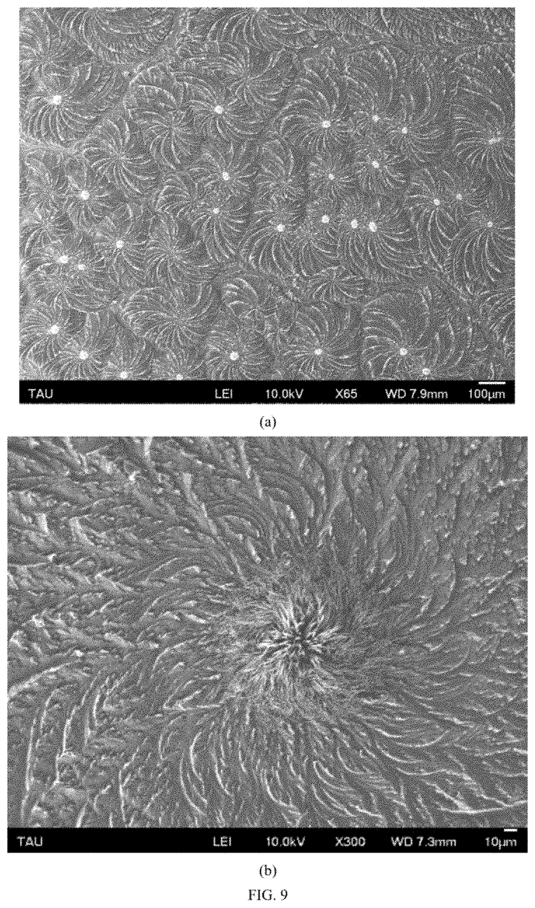

FIG. 8 was an SEM image of an amino acid film deposited on a glass substrate by a solvent evaporation method. It can be seen from the SEM image, the amino acid film prepared in the comparative example did not have a helical structure, which indicated that the amino acid helical array disclosed in the present disclosure must be prepared from terminal group-protected amino acids by a vacuum evaporation preparation technology.

### Example 21

The preparation process was substantially the same as that in example 1. A difference only lied in that a raw material was replaced with an equal mass of t-butoxycarbonyl-(D-)phenylalanine-nitrophenyl ester powder.

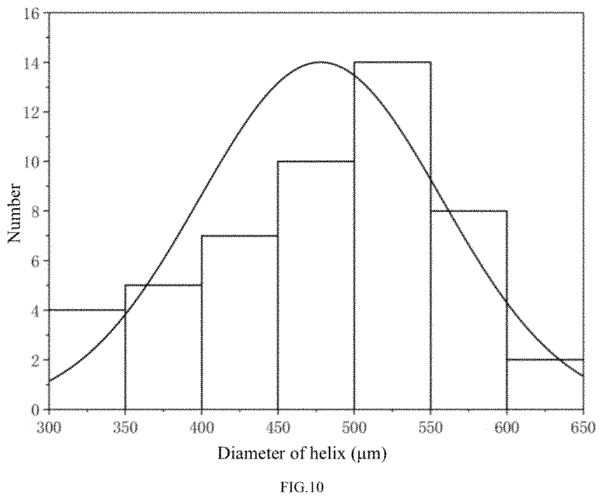

FIG. 9 was SEM images of the amino acid self-assembled helical array prepared in the example at different magnification times. It can be observed from (a) that an amino acid self-assembly formed a helical array structure. It can be observed from (b) that the helical arrays all rotated in an anticlockwise with the consistent rotation direction which was just opposite to that of the amino acid helical array prepared in example 1. The rotation directions were in a chiral symmetry.

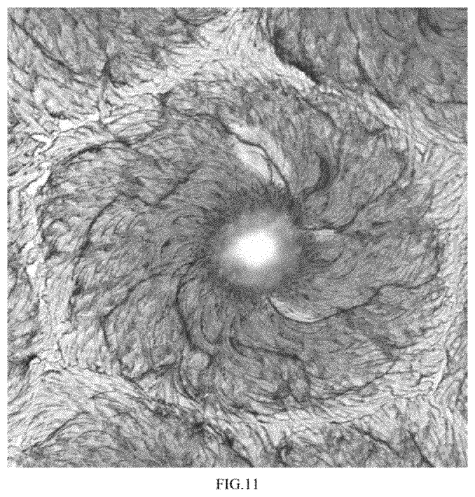

FIG. 10 was a statistical distribution diagram of diameters of the amino acid self-assembled helical array prepared in the example. The statistical result showed that the amino acid helical array had a statistical average diameter of 550+/−42 μm, a size close to that in example 1, a narrow diameter distribution, and a relatively uniform diameter.

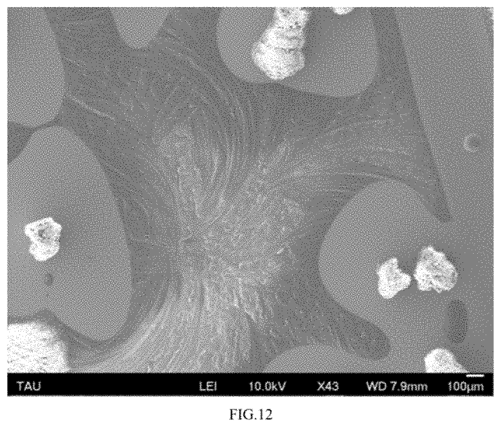

FIG. 11 was a confocal microscopy image of the amino acid self-assembled helical array prepared in the example. From the figure, it can be seen that crystal fibers at a center of helixes were tightly clustered and then spread out in an arrangement along an anticlockwise helix rotation direction.

It was indicated that the amino acid helical array disclosed in the present disclosure may adjust the rotation direction of the amino acid by controlling chirality of the amino acid.

### Comparative Example 4

The preparation process was substantially the same as that in example 1. A difference only lied in that a raw material was replaced with a mixture of equal mass of t-butoxycarbonyl-(L-)phenylalanine-nitrophenyl ester powder and t-butoxycarbonyl-(D-)phenylalanine-nitrophenyl ester powder with a total mass of 5 mg.

FIG. 12 was an SEM image of the amino acid self-assembled array film prepared in the comparative example. From the SEM image, the amino acid self-assembled array prepared in the example was a random, irregular, and non-uniform film, indicating that the amino acid helical array disclosed in the present disclosure must be prepared from a modified amino acid of a single chirality.

1. An amino acid helical array film, comprising a substrate and an amino acid helical array uniformly deposited on the substrate;
wherein each amino acid helix is obtained by self-assembling an amino acid with a modifying group;
the amino acid is selected from glycine, alanine, valine, leucine, isoleucine, methionine, proline, tryptophan, serine, tyrosine, cysteine, phenylalanine, asparagine, glutamine, threonine, aspartic acid, glutamic acid, lysine, arginine, histidine, or one or more of adjacent isomers of the above amino acids; and
the modifying group comprises an N-terminal protecting group and a C-terminal protecting group, wherein the N-terminal protecting group is selected from one or more of carbobenzoxy, a lipid group, t-butoxycarbonyl, and 9-fluorenylmethoxycarbonyl, and the C-terminal protecting group is selected from one or more of nitrophenyl ester, a lipoxy group, and an acylamino group.

2. The amino acid helical array film according to claim 1, wherein
the amino acid helical array has a uniform rotation direction in a clockwise or an anticlockwise manner; and
the amino acid helix has a diameter range of 300-650 μm.

3. The amino acid helical array film according to claim 1, wherein a material of the substrate is selected from conductive or insulating, transparent or opaque, thermally conductive, organic or inorganic, flexible or rigid metal, glass or polymer.

4. The amino acid helical array film according to claim 1, wherein the amino acid is selected from L-phenylalanine or D-phenylalanine, the N-terminal protecting group is selected from t-butoxycarbonyl, and the C-terminal protecting group is selected from nitrophenyl ester.

5. A preparation method of the amino acid helical array film according to claim 1, wherein a physical vapor deposition is used and specifically comprises:
placing an amino acid raw material with a modifying group in an evaporation boat of a reaction chamber and obtaining a self-assembled amino acid helical array film by deposition on a surface of the substrate through a vacuum evaporation coating method.

6. The preparation method of the amino acid helical array film according to claim 5, wherein a distance between the evaporation boat and the substrate is 1-5 cm.

7. The preparation method of the amino acid helical array film according to claim 5, wherein the vacuum evaporation coating method is as follows:
vacuumizing the reaction chamber until a vacuum degree is less than or equal to 5×10⁻⁶ mbar, firstly performing heating to a sublimation temperature of the amino acid raw material with a modifying group, then performing heating to a highest temperature, and preserving the temperature for a period of time;
the highest temperature is 200-220° C.; and
total time from the heating to the sublimation temperature to the end of the highest temperature preserving is recorded as deposition time, selected from 15-60 min.

8. The preparation method of the amino acid helical array film according to claim 7, wherein the deposition time is selected from 30-60 min.

9. The preparation method of the amino acid helical array film according to claim 5, the amino acid raw material with a modifying group is selected from t-butyloxycarboryl-L-phenylalanine-nitrophenyl ester or t-butyloxycarboryl-D-phenylalanine-nitrophenyl ester.
